(19) United States
(12) Patent Application Publication
(10) Pub. No.: US 2017/0356015 A1
(43) Pub. Date: Dec. 14, 2017

(54) METHOD FOR PRETREATING WOOD DUST AND METHOD FOR MANUFACTURING BIOALCOHOL

(71) Applicants: NATIONAL TSING HUA UNIVERSITY, HSINCHU (TW); Chang Chun Plastics Co., Ltd., TAIPEI (TW); Chang Chun Petrochemical Co., LTD., TAIPEI (TW)

(72) Inventors: Chung-Sung TAN, Hsinchu City (TW); The Duy PHAN, HSINCHU (TW)

(21) Appl. No.: 15/372,412

(22) Filed: Dec. 8, 2016

(30) Foreign Application Priority Data
Jun. 13, 2016 (TW) 105118433

(51) Int. Cl. C12P 7/20; C12P 7/16; C12P 7/10

(52) U.S. Cl. CPC C12P 7/20; C12P 7/10; C12P 7/16; C12P 2203/00

(57) ABSTRACT

A method for pretreating a wood dust includes conducting a structurally damaged step and an alkali treatment step. In the structurally damaged step, the wood dust is disposed in a scCO₂ atmosphere with a pressure of 2600 psi to 3400 psi at a temperature of 40° C. to 120° C. for a predetermined time, and then the pressure is adjusted to drop to an atmospheric pressure in a sudden manner to obtain a structurally damaged wood dust. In the alkali treatment step, the structurally damaged wood dust is immersed in an alkaline H₂O₂ solution at a temperature of 50° C. to 70° C., a concentration of H₂O₂ in the alkaline hydrogen peroxide solution is in a range of 0.1 wt % to 2.1 wt %, and a pH value of the alkaline H₂O₂ solution is in a range of 10.5 to 12. Thus a treated wood dust is obtained.

### RELATED APPLICATIONS

This application claims priority to Taiwan Application Serial Number 105118433, filed Jun. 13, 2016, which is herein incorporated by reference.

### BACKGROUND

### Technical Field

The present disclosure relates to a pretreatment method and a method for manufacturing bioalcohol. More particularly, the present disclosure relates to a method for pretreating wood dust and method for manufacturing bioalcohol using the same.

### Description of Related Art

With industrialization, the dependence of human beings on energy is growing. However, the reserves of non-renewable energy sources, such as petroleum, coal and natural gas, are depleting. Using bioalcohols, such as ethanol, glycerol and butanol, as alternative energy sources is one of the strategies for reducing the dependence on non-renewable energy sources.

Nowadays, the biomass for manufacturing bioalcohol mainly includes sugar, starch and lignocellulose. The biomass which provides the sugar is mainly sugarcane. The biomass which provides the starch includes corn, cassava and grain. The biomass which provides the lignocellulose includes wheat straw, rice straw, corn cobs and bagasse. Techniques for manufacturing bioalcohol with the sugar and starch are well developed. However, manufacturing bioalcohol with the sugar and starch tends to compete with food resources and results in a higher food price. Furthermore, there is limited land suitable for cultivation of crops for producing sugar and starch on earth. Accordingly, manufacturing bioalcohol with the lignocellulose has become the focus of development.

The lignocellulose is mainly composed of cellulose, hemicellulose and lignin. The cellulose is a polysaccharide consisting of glucose units, and the hemicellulose is a polysaccharide consisting of various monomer units, such as glucose, xylose, galactose, arabinose and mannose. The monosaccharides of the cellulose and the hemicellulose can be converted into alcohols. However, a pretreatment is required for the lignocellulose to remove a portion of the lignin, so that the cellulose and the hemicellulose can be favorably hydrolyzed into monosaccharides, such as glucose, xylose, and the conversion of the monosaccharides into bioalcohol can be improved, too.

The pretreatment technique is critical to the saccharification rate of biomass. A high saccharification rate can help to reduce the cost and achieve mass production. However, different biomass has a different composition ratio of the cellulose, hemicellulose and lignin. Accordingly, the existing pretreatment techniques cannot be directly applied to new biomass. The known biomass which provides the lignocellulose includes wheat straw, rice straw, corn cobs and bagasse. The common features of the aforementioned biomass are having a lower content of lignin (all lower than 25 wt %) and a looser structure, so that the lignin thereof can be removed easily. However, as for a biomass with a higher content of lignin and a denser structure, there still lacks of a pretreatment method to effectively remove the lignin thereof. Take the wood for example, the wood contains about 39.6 wt % of cellulose and 11.7 wt % of hemicellulose, which indicates that the wood is a potential candidate for manufacturing bioalcohol. However, the wood has the content of the lignin up to 39 wt % and a dense structure. It is difficult to spoil the fiber structure of the wood so as to remove the lignin. Multiple attempts have been made, but none can successfully use the wood to manufacture the bioalcohol. Therefore, how to provide an effective method for pretreating the biomass with a higher content of lignin and a denser structure so as to improve the development of the bioalcohol has become an important goal of relevant academia and industry.

### SUMMARY

According to one aspect of the present disclosure, a method for pretreating a wood dust includes steps as follows. A structurally damaged step is conducted, wherein the wood dust is disposed in a supercritical carbon dioxide (scCO₂) atmosphere with a pressure of 2600 psi to 3400 psi at a temperature of 40° C. to 120° C. for a predetermined time, and then the pressure is adjusted to drop to an atmospheric pressure in a sudden manner to obtain a structurally damaged wood dust. An alkali treatment step is conducted, wherein the structurally damaged wood dust is immersed in an alkaline hydrogen peroxide (H₂O₂) solution at a temperature of 50° C. to 70° C., a concentration of H₂O₂ in the alkaline H₂O₂ solution is in a range of 0.1 wt % to 2.1 wt %, and a pH value of the alkaline H₂O₂ solution is in a range of 10.5 to 12. Thus a treated wood dust is obtained.

According to another aspect of the present disclosure, a method for manufacturing a bioalcohol includes steps as follows. A pretreatment step, a hydrolysis step and a fermentation step are conducted. The pretreatment step includes steps as follows. A structurally damaged step is conducted, wherein the wood dust is disposed in a scCO₂ atmosphere with a pressure of 2600 psi to 3400 psi at a temperature of 40° C. to 120° C. for a predetermined time, and then the pressure is adjusted to drop to an atmospheric pressure in a sudden manner to obtain a structurally damaged wood dust. An alkali treatment step is conducted, wherein the structurally damaged wood dust is immersed in an alkaline H₂O₂ solution at a temperature of 50° C. to 70° C., a concentration of H₂O₂ in the alkaline H₂O₂ solution is in a range of 0.1 wt % to 2.1 wt %, and a pH value of the alkaline H₂O₂ solution is in a range of 10.5 to 12. Thus a treated wood dust is obtained. In the hydrolysis step, a polysaccharide of the treated wood dust is hydrolyzed into monosaccharides. In the fermentation step, the monosaccharides are converted into an alcohol.

### BRIEF DESCRIPTION OF THE DRAWINGS

The present disclosure can be more fully understood by reading the following detailed description of the embodiment, with reference made to the accompanying drawings as follows:

FIG. 1 is a flow diagram showing a method for pretreating a wood dust according to one embodiment of the present disclosure;

FIG. 2 is a flow diagram showing a method for pretreating a wood dust according to another embodiment of the present disclosure;

FIG. 3 is a flow diagram showing a method for manufacturing a bioalcohol according to yet another embodiment of the present disclosure;

FIG. 4 is a flow diagram showing Step 310 in FIG. 3;

FIG. 5 is a flow diagram showing Step 310 according to further another embodiment of the present disclosure;

FIG. 6 are scanning electron microscope (SEM) results of the comparative example (Com Ex.) 1 to Com Ex. 3 and example (Ex.) 1; and

FIG. 7 shows relationships of glucose recovery and time of Ex. 1 to Ex. 4 and Com Ex. 1 to Com Ex. 6.

### DETAILED DESCRIPTION

### Method for Pretreating Wood Dust

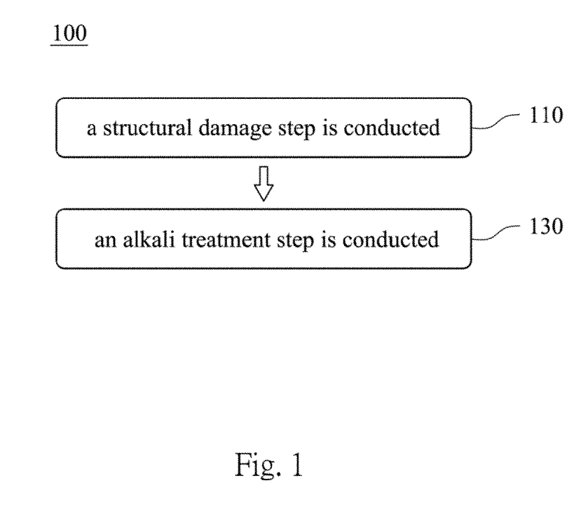

FIG. 1 is a flow diagram showing a method for pretreating a wood dust 100 according to one embodiment of the present disclosure. In FIG. 1, the method for pretreating the wood dust 100 includes Step 110 and Step 130.

In Step 110, a structurally damaged step is conducted. The wood dust is disposed in a scCO₂ atmosphere with a pressure of 2600 psi to 3400 psi at a temperature of 40° C. to 120° C. for a predetermined time, and then the pressure is adjusted to drop to an atmospheric pressure in a sudden manner to obtain a structurally damaged wood dust. By Step 110, it is favorable for the scCO₂ permeating into the fiber structure of the wood dust so as to spoil the fiber structure of the wood dust. Accordingly, the dissolving efficiency of the lignin in the following alkali treatment step can be enhanced.

The aforementioned “in a sudden manner” refers that the pressure can drop to the atmospheric pressure within 3 seconds.

In Step 130, an alkali treatment step is conducted. The structurally damaged wood dust is immersed in an alkaline H₂O₂ solution at a temperature of 50° C. to 70° C., a concentration of H₂O₂ in the alkaline H₂O₂ solution is in a range of 0.1 wt % to 2.1 wt %, and a pH value of the alkaline H₂O₂ solution is in a range of 10.5 to 12, thus a treated wood dust is obtained. Therefore, a portion of the lignin in the wood dust can be removed, which is favorable for hydrolyzing the polysaccharide (i.e., the cellulose and the hemicellulose) bonded with the lignin into monosaccharides.

With the method for pretreating the wood dust 100, the glucose recovery of the treated wood dust can be significantly enhanced, which makes it possible to apply the wood dust to manufacture bioalcohol in practical use. Furthermore, the operating temperature of the method for pretreating the wood dust 100 (40° C. to 120° C. and 50° C. to 70° C.) can avoid the degradation of hemicellulose and the formation of furfural. The degradation of hemicellulose will inhibit the hydrolysis rate in the subsequent hydrolysis step, and the formation of furfural will inhibit the fermentation rate in the fermentation step.

Specifically, in Step 110, the wood dust can recycle waste wood dust or waste wood as raw material, which can reduce the burden of dealing with the waste wood dust and the waste wood, and can meet the environmental demands. Furthermore, a particle size of the wood dust can be in a range of 5 μm to 10 μm. Accordingly, the time required for the method for pretreating the wood dust 100 can be reduced. The wood and the wood dust with a larger particle size can be processed with equipment such as wood chipper or grinder, so that the desired particle size can be obtained. How to process the wood and the wood dust so as to obtain the desired particle size is conventional, and will not be repeated herein.

In Step 110, the predetermined time can be 7.5 minutes to 37.5 minutes. Therefore, it is favorable for the scCO₂ permeating into the fiber structure of the wood dust, and then the pressure is adjusted to drop to the atmospheric pressure in a sudden manner.

In Step 130, the alkaline H₂O₂ solution is prepared by mixing H₂O₂ and deionized water or distilled water, and then the pH value thereof is adjusted by adding NaOH.

Step 130 can be conducted at most 9 hours. Therefore, a desired amount of lignin can be removed.

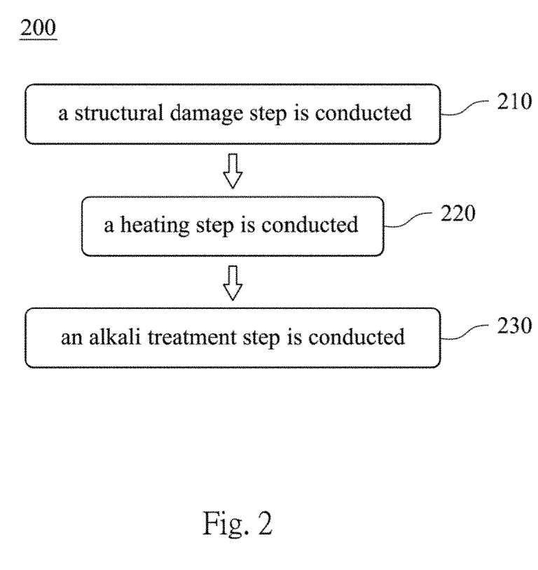

FIG. 2 is a flow diagram showing a method for pretreating a wood dust 200 according to another embodiment of the present disclosure. In FIG. 2, the method for pretreating the wood dust 200 includes Step 210, Step 220 and Step 230. Comparing to the method for pretreating the wood dust 100 in FIG. 1, there is one more Step 220 in the method for pretreating the wood dust 200.

In Step 220, a heating step is conducted before conducting the alkali treatment step. The structurally damaged wood dust is heated with a temperature of 80° C. to 120° C. Moreover, Step 220 can be conducted for 15 minutes to 30 minutes. Therefore, the structurally damaged effect can be improved, and the glucose recovery can be further enhanced.

Step 210 can be the same as Step 110 in FIG. 1, and Step 230 can be the same as Step 130 in FIG. 1. Therefore, Step 210 and Step 230 will not be repeated herein.

### Method for Manufacturing Bioalcohol

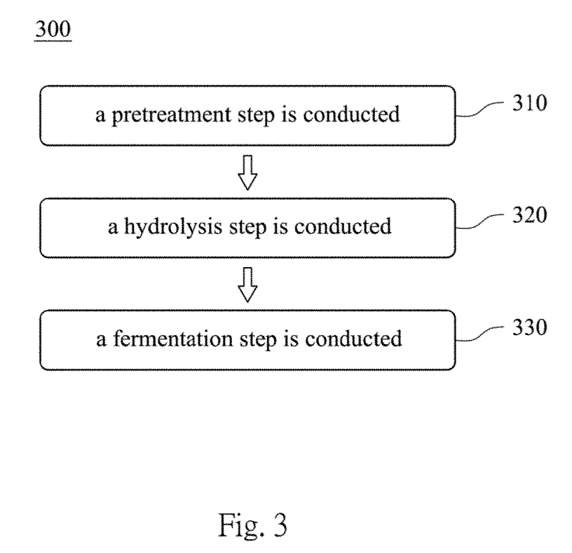

FIG. 3 is a flow diagram showing a method for manufacturing a bioalcohol 300 according to yet another embodiment of the present disclosure. In FIG. 3, the method for manufacturing the bioalcohol 300 includes Step 310, Step 320 and Step 330.

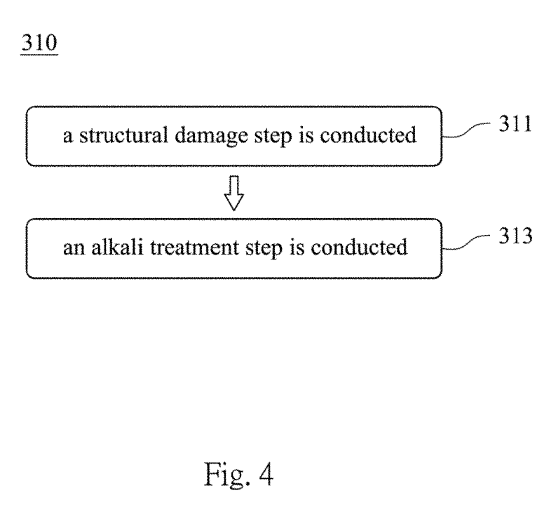

In Step 310, a pretreatment step is conducted. FIG. 4 is a flow diagram showing Step 310 in FIG. 3. As shown in FIG. 4, Step 310 includes Step 311 and Step 313. In Step 311, a structurally damaged step is conducted. In Step 313, an alkali treatment step is conducted. Step 311 can be the same as Step 110 in FIG. 1, and Step 313 can be the same as Step 130 in FIG. 1. Therefore, Step 311 and Step 313 will not be repeated herein. By Step 310, a treated wood dust is obtained.

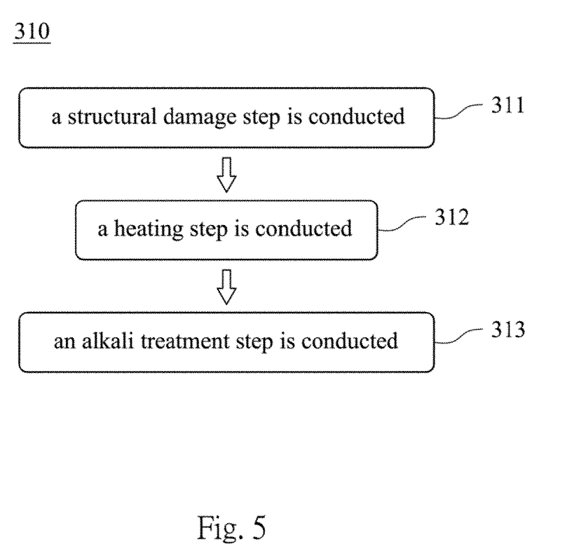

FIG. 5 is a flow diagram showing Step 310 according to further another embodiment of the present disclosure. In FIG. 5, Step 310 includes Step 311, Step 312 and Step 313. Comparing to Step 310 of FIG. 4, there is one more Step 312 in FIG. 5. In Step 312, a heating step is conducted. Step 312 can be the same as Step 220 in FIG. 2, and will not be repeated herein.

In Step 320, a hydrolysis step is conducted, in which a polysaccharide of the treated wood dust is hydrolyzed into monosaccharides.

In Step 330, a fermentation step is conducted, in which the monosaccharides are converted into an alcohol.

Specifically, Step 320 can be an acid hydrolysis step or an enzyme hydrolysis step. How to hydrolyze the polysaccharide into monosaccharides is conventional, and will not be repeated herein.

Specifically, the alcohol in Step 330 can be ethanol, glycerol or butanol. The kinds of enzyme in the fermentation step can be decided according to the desired kinds of the alcohol.

With the wood dust as the biomass, the method for manufacturing the bioalcohol 300 can broadened the source of biomass to the wood, which is featured with a higher content of lignin. Therefore, it is favorable to improve the development of the bioalcohol, and can reduce the dependence on the non-renewable energy sources.

### EXAMPLES

Ex. 1: wood dust with a particle size in a range of 5 μm to 10 μm (5 g) is disposed in a reaction container. CO₂ is introduced into the reaction container and is heated to 80° C. and pressurized to 2800 psi, so that the wood dust is disposed in a scCO₂ atmosphere. After 15 minutes, the pressure is adjusted to drop to an atmospheric pressure in a sudden manner to obtain a structurally damaged wood dust. The atmospheric pressure is about 1 atm. The structurally damaged wood dust is immersed in an alkaline H₂O₂ solution at a temperature of 60° C. for 9 hours. A concentration of H₂O₂ in the alkaline H₂O₂ solution is in a range of 1 wt % to 1.6 wt %, and a pH value of the alkaline H₂O₂ solution is 11.5. Thus the treated wood dust of Ex. 1 is obtained.

Ex. 2: wood dust with a particle size in a range of 5 μm to 10 μm (5 g) is disposed in a reaction container. CO₂ is introduced into the reaction container and is heated to 80° C. and pressurized to 2800 psi, so that the wood dust is disposed in a scCO₂ atmosphere. After 15 minutes, the pressure is adjusted to drop to the atmospheric pressure in a sudden manner to obtain a structurally damaged wood dust. The structurally damaged wood dust is heated with a temperature of 80° C. under the atmospheric pressure for 15 minutes. The structurally damaged wood dust is then immersed in an alkaline H₂O₂ solution at a temperature of 60° C. for 9 hours. A concentration of H₂O₂ in the alkaline H₂O₂ solution is in a range of 1 wt % to 1.6 wt %, and a pH value of the alkaline H₂O₂ solution is 11.5. Thus the treated wood dust of Ex. 2 is obtained.

Ex. 3: the structurally damaged wood dust is heated with a temperature of 100° C. under the atmospheric pressure for 15 minutes, and other steps are the same as that of Ex. 2. Thus the treated wood dust of Ex. 3 is obtained.

Ex. 4: the structurally damaged wood dust is heated with a temperature of 120° C. under the atmospheric pressure for 15 minutes, and other steps are the same as that of Ex. 2. Thus the treated wood dust of Ex. 4 is obtained.

Ex. 5 to Ex. 35: the conditions of the structurally damaged step, the heating step and the alkali treatment step of Ex. 2 are changed as shown in Table 1, and other steps are the same as that of Ex. 2. Thus the treated wood dust of Ex. 5 to Ex. 35 are obtained. In Table 1, “T1” represents the temperature in the structurally damaged step, “P” represents the pressure in the structurally damaged step, “t1” represents the predetermined time in the structurally damaged step, “T2” represents the temperature in the heating step, “t2” represents the time for the heating step, “C” represents the concentration of H₂O₂ in the alkaline H₂O₂ solution, “t3” represents the time for the alkali treatment step and “T3” represents the temperature in the alkali treatment step.

TABLE 1

| | structurally damaged | | | heating | | | | | |
|---|---|---|---|---|---|---|---|---|---|
| | step | | | step | | alkali treatment step | | | |
| | T1 | P | t1 | T2 | t2 | C | | t3 | T3 |
| EX. | (° C.) | (psi) | (min) | (° C.) | (min) | (wt %) | pH | (hr) | (° C.) |
| 5 | 60 | 2800 | 15 | 100 | 15 | 0.6 | 11.5 | 9 | 60 |
| 6 | 100 | 2800 | 15 | 100 | 15 | 0.6 | 11.5 | 9 | 60 |
| 7 | 60 | 3200 | 15 | 100 | 15 | 0.6 | 11.5 | 9 | 60 |
| 8 | 100 | 3200 | 15 | 100 | 15 | 0.6 | 11.5 | 9 | 60 |
| 9 | 60 | 2800 | 30 | 100 | 15 | 0.6 | 11.5 | 9 | 60 |
| 10 | 100 | 2800 | 30 | 100 | 15 | 0.6 | 11.5 | 9 | 60 |
| 11 | 60 | 3200 | 30 | 100 | 15 | 0.6 | 11.5 | 9 | 60 |
| 12 | 100 | 3200 | 30 | 100 | 15 | 0.6 | 11.5 | 9 | 60 |
| 13 | 60 | 2800 | 15 | 100 | 15 | 1.6 | 11.5 | 9 | 60 |
| 14 | 100 | 2800 | 15 | 100 | 15 | 1.6 | 11.5 | 9 | 60 |
| 15 | 60 | 3200 | 15 | 100 | 15 | 1.6 | 11.5 | 9 | 60 |
| 16 | 100 | 3200 | 15 | 100 | 15 | 1.6 | 11.5 | 9 | 60 |
| 17 | 60 | 2800 | 30 | 100 | 15 | 1.6 | 11.5 | 9 | 60 |
| 18 | 100 | 2800 | 30 | 100 | 15 | 1.6 | 11.5 | 9 | 60 |
| 19 | 60 | 3200 | 30 | 100 | 15 | 1.6 | 11.5 | 9 | 60 |
| 20 | 100 | 3200 | 30 | 100 | 15 | 1.6 | 11.5 | 9 | 60 |
| 21 | 40 | 3000 | 22.5 | 100 | 15 | 1.1 | 11.5 | 9 | 60 |
| 22 | 120 | 3000 | 22.5 | 100 | 15 | 1.1 | 11.5 | 9 | 60 |
| 23 | 80 | 2600 | 22.5 | 100 | 15 | 1.1 | 11.5 | 9 | 60 |
| 24 | 80 | 3400 | 22.5 | 100 | 15 | 1.1 | 11.5 | 9 | 60 |
| 25 | 80 | 3000 | 7.5 | 100 | 15 | 1.1 | 11.5 | 9 | 60 |
| 26 | 80 | 3000 | 37.5 | 100 | 15 | 1.1 | 11.5 | 9 | 60 |
| 27 | 80 | 3000 | 22.5 | 100 | 15 | 0.1 | 11.5 | 9 | 60 |
| 28 | 80 | 3000 | 22.5 | 100 | 15 | 2.1 | 11.5 | 9 | 60 |
| 29 | 80 | 3000 | 22.5 | 100 | 15 | 1.1 | 11.5 | 9 | 60 |
| 30 | 80 | 3000 | 22.5 | 100 | 15 | 1.1 | 11.5 | 9 | 60 |
| 31 | 80 | 3000 | 22.5 | 100 | 15 | 1.1 | 11.5 | 9 | 60 |
| 32 | 80 | 3000 | 22.5 | 100 | 15 | 1.1 | 11.5 | 9 | 60 |
| 33 | 80 | 3000 | 22.5 | 100 | 15 | 1.1 | 11.5 | 9 | 60 |
| 34 | 80 | 3000 | 22.5 | 100 | 15 | 1.1 | 11.5 | 9 | 60 |
| 35 | 80 | 3000 | 22.5 | 100 | 15 | 1.1 | 11.5 | 9 | 60 |

Com Ex. 1: wood dust with a particle size in a range of 5 μm to 10 μm (5 g). That is, Com Ex. 1 is an untreated wood dust.

Com Ex. 2: wood dust with a particle size in a range of 5 μm to 10 μm (5 g) is immersed in an alkaline H₂O₂ solution at a temperature of 60° C. for 9 hours. A concentration of H₂O₂ in the alkaline H₂O₂ solution is in a range of 1 wt % to 1.6 wt %, and a pH value of the alkaline H₂O₂ solution is 11.5. Thus the treated wood dust of Com Ex. 2 is obtained.

Com Ex. 3: wood dust with a particle size in a range of 5 μm to 10 μm (5 g) is disposed in a reaction container. CO₂ is introduced into the reaction container and is heated to 80° C. and pressurized to 2800 psi, so that the wood dust is disposed in a scCO₂ atmosphere. After 15 minutes, the pressure is adjusted to drop to the atmospheric pressure in a sudden manner. Thus the treated wood dust of Com Ex. 3 is obtained.

Com Ex. 4: wood dust with a particle size in a range of 5 μm to 10 μm (5 g) is disposed in a reaction container. CO₂ is introduced into the reaction container and is heated to 80° C. and pressurized to 2800 psi, so that the wood dust is disposed in a scCO₂ atmosphere. After 15 minutes, the pressure is adjusted to drop to the atmospheric pressure in a sudden manner to obtain a structurally damaged wood dust. The structurally damaged wood dust is heated with a temperature of 80° C. under the atmospheric pressure for 15 minutes. Thus the treated wood dust of Com Ex. 4 is obtained.

Com Ex. 5: the structurally damaged wood dust is heated with a temperature of 100° C. under the atmospheric pressure for 15 minutes, and other steps are the same as that of Com Ex. 4. Thus the treated wood dust of Com Ex. 5 is obtained.

Com Ex. 6: the structurally damaged wood dust is heated with a temperature of 120° C. under the atmospheric pressure for 15 minutes, and other steps are the same as that of Com Ex. 4. Thus the treated wood dust of Com Ex. 6 is obtained.

### Compositions and Delignification Rates of Examples and Comparative Examples

The compositions of Com Ex. 1, Com Exs. 3-6 and Exs. 1-35 are obtained by acid hydrolysis method as follows. Weigh 0.3 g of the wood dust (the untreated wood dust of Com Ex. 1 or the treated wood dust of Com Exs. 3-6 and Exs. 1-35) into a tared pressure tube and then add 3 mL of 72 wt % H₂SO₄ solution therein to form a sample. Place the tared pressure tube in a water bath set at 30° C. and incubate the sample for 60 minutes. Use a stirring rod to stir the sample without removing the tared pressure tube from the water bath. Afterwards, dilute the sample to a 4 wt % concentration by adding 84 mL deionized water. Securely screw the tared pressure tube with a teflon cap, and then place the tared pressure tube in an autoclave at a temperature of 121° C. for 1 hour. Use the liquid fraction to determine glucose and xylose by HPLC (High Performance Liquid Chromatography) and convert to cellulose and hemicellulose. Use the solid fraction to determine the acid insoluble lignin. The delignification rate is calculated by the measured results. The compositions and delignification rates of Com Ex. 1, Com Exs. 3-6 and Exs. 1-35 are listed in Table 2.

TABLE 2

| Com Ex./ | composition | | | delignification |
|---|---|---|---|---|
| Ex. | cellulose (%) | hemicellulose (%) | lignin (%) | rate (%) |
| Com Ex. 1 | 39.6 | 11.7 | 39.4 | — |
| Com Ex. 3 | 41.4 | 11.8 | 39.2 | 0.51 |
| Com Ex. 4 | 41.3 | 12.9 | 39.3 | 0.25 |
| Com Ex. 5 | 41.8 | 11.2 | 39.2 | 0.51 |
| Com Ex. 6 | 41.9 | 11.2 | 39.2 | 0.51 |
| Ex. 1 | 50.8 | 11.3 | 33.8 | 14.2 |
| Ex. 2 | 51.8 | 11.3 | 31.9 | 19.0 |
| Ex. 3 | 51.9 | 11.5 | 31.2 | 20.8 |
| Ex. 4 | 52.0 | 11.4 | 31.1 | 21.1 |
| Ex. 5 | 50.5 | 13.7 | 33.5 | 15.0 |
| Ex. 6 | 50.7 | 13.8 | 33.6 | 14.7 |
| Ex. 7 | 49.2 | 13.1 | 33.6 | 14.7 |
| Ex. 8 | 49.1 | 13.6 | 33.7 | 14.5 |
| Ex. 9 | 53.1 | 16.9 | 33.9 | 14.0 |
| Ex. 10 | 52.1 | 12.2 | 32.4 | 17.8 |
| Ex. 11 | 52.1 | 12.2 | 32.6 | 17.3 |
| Ex. 12 | 51.8 | 12.5 | 32.0 | 18.8 |
| Ex. 13 | 56.2 | 12.1 | 33.8 | 14.2 |
| Ex. 14 | 54.6 | 12.1 | 31.8 | 19.3 |
| Ex. 15 | 54.6 | 11.5 | 34.1 | 13.5 |
| Ex. 16 | 53.2 | 12.0 | 31.7 | 19.5 |
| Ex. 17 | 57.1 | 13.4 | 35.7 | 9.4 |
| Ex. 18 | 56.8 | 16.4 | 32.8 | 16.8 |
| Ex. 19 | 55.5 | 11.9 | 34.7 | 11.9 |
| Ex. 20 | 56.5 | 15.8 | 31.8 | 19.3 |
| Ex. 21 | 52.2 | 11.6 | 33.9 | 14.0 |
| Ex. 22 | 51.3 | 11.7 | 33.7 | 14.5 |
| Ex. 23 | 53.1 | 11.6 | 33.5 | 15.0 |
| Ex. 24 | 51.3 | 11.5 | 33.8 | 14.2 |
| Ex. 25 | 53.9 | 11.9 | 34.1 | 13.5 |
| Ex. 26 | 52.2 | 11.4 | 33.8 | 14.2 |
| Ex. 27 | 50.7 | 12.0 | 34.3 | 12.9 |
| Ex. 28 | 54.7 | 11.2 | 31.8 | 19.3 |
| Ex. 29 | 51.9 | 11.8 | 31.7 | 19.5 |
| Ex. 30 | 52.8 | 12.4 | 33.1 | 16.0 |
| Ex. 31 | 54.9 | 14.2 | 32.5 | 17.5 |
| Ex. 32 | 52.9 | 11.5 | 32.6 | 17.3 |
| Ex. 33 | 53.5 | 11.7 | 32.8 | 16.8 |
| Ex. 34 | 55.8 | 14.0 | 31.8 | 19.3 |
| Ex. 35 | 52.9 | 12.0 | 32.4 | 17.8 |

Note:
delignification rate (%) = 100 × (1 − X/Y).
X represents the composition ratio of the lignin of Com Exs. 3-6 and Ex. 1-35.
Y represents the composition ratio of the lignin of Com Ex1.

As shown in Table 2, the method for pretreating the wood dust according to the present disclosure can enhance the delignification rate. Moreover, as shown in Exs. 1-4, when the other conditions are the same, adding the heating step can enhance the effect of delignification.

### Specific Surface Area

The specific surface areas of Com Ex. 1, 2, 5 and Ex. 1 are obtained by a Brunauer-Emmett-Teller (BET) measurement with Autosorb-1 (purchased from Quantachrome Instrument), which can obtain the adsorption and desorption isotherm of nitrogen. Moreover, the pore volume and pore size are measured, too. The results are listed in Table 3.

TABLE 3

| Com Ex./ | specific surface areas | pore volume | pore size |
|---|---|---|---|
| Ex. | (m²/g) | (cm³/g) | (Å) |
| Com Ex. 1 | 1.44 | 0.006 | 165 |
| Com Ex. 2 | 0.96 | 0.004 | 162 |
| Com Ex. 5 | 1.96 | 0.007 | 189 |
| Ex. 1 | 2.01 | 0.008 | 205 |

As shown in Table 3, the method for pretreating the wood dust according to the present disclosure can enhance the specific surface area, pore volume and pore size, which is favorable to enhance the glucose recovery.

### SEM Results

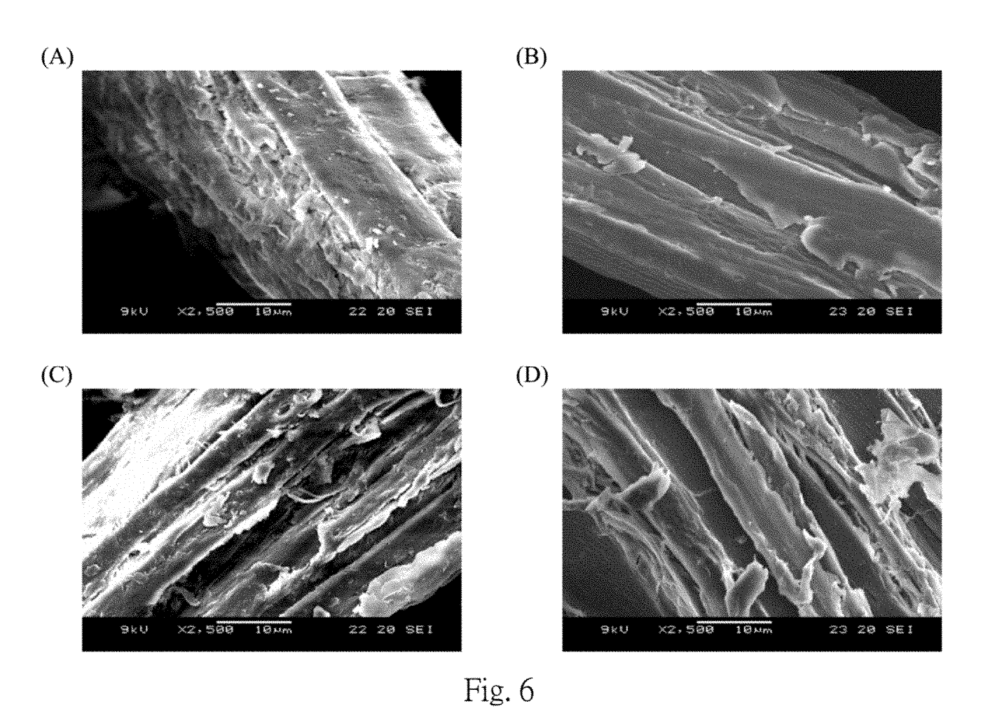

The untreated wood dust of Com Ex. 1 and the treated wood dust of Com Ex. 2-3 and Ex. 1 are observed by a SEM (JSM-5600, purchased from JEOL). FIG. 6 are SEM results of Com Ex. 1-3 and Ex. 1, in which (A) is the SEM result of Com Ex. 1, (B) is the SEM result of Com Ex. 2, (C) is the SEM result of Com Ex. 3, and (D) is the SEM result of Ex. 1. As shown in FIG. 6, the method for pretreating the wood dust according to the present disclosure can effectively spoil the fiber structure of the wood dust.

### Glucose Recovery

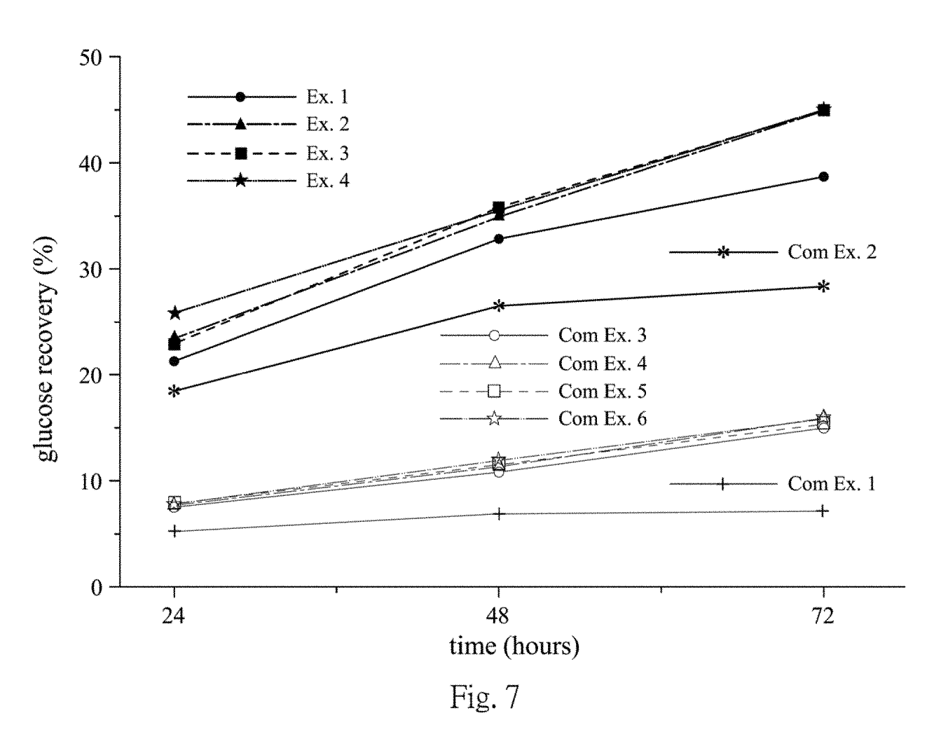

The glucose recovery at 24 hours, 48 hours and 72 hours of the untreated wood dust of Com Ex. 1 and the treated wood dust of Com Ex. 2-6 and Exs. 1-4 are measured as follows. Weigh 0.5 g of wood dust (the untreated wood dust of Com Ex. 1 or the treated wood dust of Com Exs. 2-6 and Exs. 1-4) into a 20 mL glass vial, and add 5.0 mL of sodium citrate buffer (0.1M, pH 4.8). Then add 0.1 mL of sodium azide solution (2 wt %) to prevent the growth of bacterial during the hydrolysis. Add 9.7 mL of deionized water and an appropriate volume of the CTec-2 enzyme (Novozym with 120 FPU (Filter Paper Unit)/mL) preparation to equal approximately 15 FPU/g cellulose. Close the glass vial tightly and place it in a vial rack suitable for the shaking oven. Set the temperature to 50° C. and incubate with shaking sufficiently to keep solids in constant suspension for a period of 72 hours. A 0.5 mL of an aliquot of the enzymatic hydrolysis liquid is removed at each 24 hours interval. The 0.5 mL of an aliquot of the enzymatic hydrolysis liquid is filtered through a 0.22 μm filter and subjected to glucose analysis using HPLC method. Glucose recovery (%, w/w)={(glucose in enzymatic hydrolysis liquid)/[(cellulose in the wood dust of comparative example or example)×0.9]}×100%. FIG. 7 shows relationships of glucose recovery and time of Ex. 1 to Ex. 4 and Com Ex. 1 to Com Ex. 6. As shown in FIG. 7, the glucose recovery at 72 hours of the untreated wood dust of Com Ex. 1 is only 7.1%. When the wood dust is only treated with the alkali treatment step, such as Com Ex. 2, the glucose recovery at 72 hours thereof is 28.3%. When the wood dust is only treated with the structurally damaged step, such as Com Ex. 3, or when the wood dust only treated with the structurally damaged step and the heating step, such as Com Exs. 4-6, the glucose recovery at 72 hours of each of the Com Exs. 3-6 is in the range of 14.9%-15.8%. However, the treated wood dust obtained by the method for pretreating the wood dust according to the present disclosure, such as Exs. 1-4, the glucose recovery at 72 hours of each of Exs. 1-4 is in the range of 44.8%-45.0%. It is apparent that the method for pretreating the wood dust according to the present disclosure can significantly enhance the glucose recovery. In other words, the method for pretreating the wood dust according to the present disclosure can effectively spoil the fiber structure of the wood dust (i.e., can break the bond between the lignin and the cellulose/hemicellulose), so that the glucose recovery can be enhanced.

The glucose recovery at 72 hours of each of the treated wood dust of Ex. 5-35 is measured, and the results are listed in Table 4.

TABLE 4

| Ex. | glucose recovery |
|---|---|
| 5 | 44.4 |
| 6 | 44.5 |
| 7 | 50.7 |
| 8 | 44.5 |
| 9 | 41.2 |
| 10 | 69.0 |
| 11 | 78.5 |
| 12 | 56.0 |
| 13 | 78.1 |
| 14 | 78.6 |
| 15 | 68.8 |
| 16 | 86.7 |
| 17 | 63.9 |
| 18 | 77.9 |
| 19 | 71.5 |
| 20 | 77.8 |
| 21 | 77.8 |
| 22 | 73.9 |
| 23 | 78.2 |
| 24 | 86.3 |
| 25 | 82.8 |
| 26 | 83.0 |
| 27 | 80.6 |
| 28 | 80.1 |
| 29 | 74.1 |
| 30 | 64.4 |
| 31 | 74.7 |
| 32 | 78.4 |
| 33 | 70.9 |
| 34 | 79.2 |
| 35 | 82.9 |

As shown in Table 4, the glucose recovery at 72 hours of each of Exs. 5-35 is in the range of 41.2%-86.7%, which can further confirm that the method for pretreating the wood dust according to the present disclosure can significantly enhance the glucose recovery.

According to the present disclosure, “saccharification” refers to the process that the polysaccharide, such as the cellulose and the hemicellulose, is hydrolyzed into monosaccharides. “Saccharification rate” refers to the ratio of the weight of the monosaccharides obtained by the hydrolysis and the weight of the polysaccharide before the hydrolysis. “Glucose recovery” refers to the process that the cellulose is hydrolyzed into the glucose.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A method for pretreating a wood dust, comprising:
conducting a structurally damaged step, wherein the wood dust is disposed in a supercritical carbon dioxide atmosphere with a pressure of 2600 psi to 3400 psi at a temperature of 40° C. to 120° C. for a predetermined time, and then the pressure is adjusted to drop to an atmospheric pressure in a sudden manner to obtain a structurally damaged wood dust; and
conducting an alkali treatment step, wherein the structurally damaged wood dust is immersed in an alkaline hydrogen peroxide solution at a temperature of 50° C. to 70° C., a concentration of hydrogen peroxide in the alkaline hydrogen peroxide solution is in a range of 0.1 wt % to 2.1 wt %, and a pH value of the alkaline hydrogen peroxide solution is in a range of 10.5 to 12, thus a treated wood dust is obtained.

2. The method for pretreating the wood dust of claim 1, further comprising:
conducting a heating step before conducting the alkali treatment step, wherein the structurally damaged wood dust is heated with a temperature of 80° C. to 120° C.

3. The method for pretreating the wood dust of claim 2, wherein the heating step is conducted for 15 minutes to 30 minutes.

4. The method for pretreating the wood dust of claim 1, wherein the predetermined time in the structurally damaged step is 7.5 minutes to 37.5 minutes.

5. The method for pretreating the wood dust of claim 1, wherein the alkali treatment step is conducted at most 9 hours.

6. A method for manufacturing a bioalcohol, comprising:
conducting a pretreatment step, comprising:
conducting a structurally damaged step, wherein a wood dust is disposed in a supercritical carbon dioxide atmosphere with a pressure of 2600 psi to 3400 psi at a temperature of 40° C. to 120° C. for a predetermined time, and then the pressure is adjusted to drop to an atmospheric pressure in a sudden manner to obtain a structurally damaged wood dust; and
conducting an alkali treatment step, wherein the structurally damaged wood dust is immersed in an alkaline hydrogen peroxide solution at a temperature of 50° C. to 70° C., a concentration of hydrogen peroxide in the alkaline hydrogen peroxide solution is in a range of 0.1 wt % to 2.1 wt %, and a pH value of the alkaline hydrogen peroxide solution is in a range of 10.5 to 12, thus a treated wood dust is obtained;
conducting a hydrolysis step, wherein a polysaccharide of the treated wood dust is hydrolyzed into monosaccharides; and conducting a fermentation step, wherein the monosaccharides are converted into an alcohol.

7. The method for manufacturing the bioalcohol of claim 6, wherein the pretreatment step further comprises:
conducting a heating step before conducting the alkali treatment step, wherein the structurally damaged wood dust is heated with a temperature of 80° C. to 120° C.

8. The method for manufacturing the bioalcohol of claim 7, wherein the heating step is conducted for 15 minutes to 30 minutes.

9. The method for manufacturing the bioalcohol of claim 6, wherein the predetermined time in the structurally damaged step is 7.5 minutes to 37.5 minutes.

10. The method for manufacturing the bioalcohol of claim 6, wherein the alkali treatment step is conducted at most 9 hours.
